(19) United States
(12) Patent Application Publication
(10) Pub. No.: US 2011/0144376 A1
(43) Pub. Date: Jun. 16, 2011

(54) DERMATOLOGICAL AGENT

(75) Inventors: Jiro Takata, Fukuoka-shi (JP); Nami Akaho, Fukuoka-shi (JP); Kazuhisa Matsunaga, Fukuoka-shi (JP); Yoshiharu Karube, Fukuoka-shi (JP); Manabu Nakashima, Fukuoka-shi (JP)

(73) Assignee: SHOWA DENKO K.K., Tokyo (JP)

(21) Appl. No.: 13/059,160

(22) PCT Filed: Aug. 20, 2008
(86) PCT No.: PCT/JP2008/064830

§ 371 Date: Feb. 15, 2011

(51) Int. Cl. C07C 229/34; C07C 271/22

(52) U.S. Cl. 560/158; 560/171; 560/173; 560/164

(57) ABSTRACT

An object of the present invention is to provide dermatological agents such as whitening agents and hair growth accelerators that contain a compound having high water solubility and a specific structure capable of releasing 2,3-dimethoxy-5-methyl-1,4-dihydroxybenzene (reduced form). A dermatological agent according to the present invention contains a 2,3-dimethoxy-5-methyl-1,4-dihydroxybenzenecarboxylic acid ester derivative represented by the following general Formula (I) or a salt thereof:

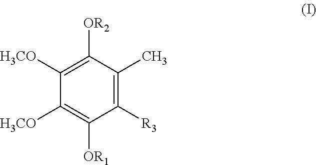

wherein in the Formula (I), R₁, R₂ and R₃ are specific groups.

### TECHNICAL FIELD

The present invention relates to improvements in dermatological agents, in particular dermatological agents containing ubiquinone analogues.

### BACKGROUND ART

A variety of medicinal components are added to dermatological agents such as emulsions, creams, lotions, packs, cleansers, dispersions, ointments, detergents, aerosols, adhesive skin patches, cataplasms and liniments to give the desired medicinal effects.

The 2,3-dimethoxy-5-methyl-1,4-benzoquinone compound that is found most abundant in mammals is ubiquinone-10. Because ubiquinone-10 has a C50 decaisoprenyl group as a side chain bonded to the benzene ring, this compound shows very high lipophilicity and is completely insoluble in water. The water insolubility of ubiquinone-10 limits possible formulations of dermatological agents in which it is blended. Ubiquinone-10 thus has formulation restrictions.

On the other hand, while ubiquinone-10 is widely distributed in cell membranes, the reduced form of ubiquinone-10, ubiquinol-10, which has high antioxidant effects and high usefulness is found in cell membranes in small amounts, approximately 1/10 compared to α-tocopherol. To obtain efficiently the antioxidant effects of 2,3-dimethoxy-5-methyl-1,4-dihydroxybenzene compound (the reduced form), effective supply of the reduced compound is desired. However, the supply of the reduced compound is dependent on the ability of reductases in the body.

Formulations using lipids such as emulsions, liposomes, microparticles and nanoparticles are known as aqueous dispersions of ubiquinone-10 (Patent Document 1, Patent Document 2, Patent Document 3 and Patent Document 4). The related conventional art further includes a patent on formulations using high-concentration surfactant, lipid and polyethylene glycol (Patent Document 5), a patent on formulations containing medium-chain fatty acid monoglyceride and plant oil (Patent Document 6) , and a mixture of ubiquinone and phospholipid (Patent Document 7).

The art described in these patent documents solves the problems associated with the difficult solubility of ubiquinone-10, by preparing aqueous dispersions of the oxidized form, i.e., 2,3-dimethoxy-5-methyl-1,4-benzoquinone compound. The problems remained for the attainment of bioavailability of the reduced form, 2,3-dimethoxy-5-methyl-1,4-dihydroxybenzene compound.

There has been then proposed a derivative of 2,3-dimethoxy-5-methyl-1,4-dihydroxybenzene compound that can solve both drug delivery problems attributed to the difficult solubility in water of 2,3-dimethoxy-5-methyl-1,4-benzoquinone compound and drug delivery problems due to the process of its reduction to 2,3-dimethoxy-5-methyl-1,4-dihydroxybenzene (the reduced form) (Patent Document 8).

The Patent Document 8 describes that the derivative releases 2,3-dimethoxy-5-methyl-1,4-dihydroxybenzene (the reduced form) in vivo, but does not disclose any medicinal effects of the compound. The document does not teach whether or not the compound shows medicinal effects as an active ingredient in dermatological preparations.

### Patent Document 1: WO 95/05164

Patent Document 2: U.S. Pat. No. 4,824,669
Patent Document 3: U.S. Pat. No. 4,636,381
Patent Document 4: U.S. Pat. No. 4,483,873

### Patent Document 5: WO 86/04503

### Patent Document 6: JP-A-S63-188623

Patent Document 7: U.S. Pat. No. 4,684,520

### Patent Document 8: JP-A-2003-104945

### SUMMARY OF INVENTION

### Technical Problem

The present invention has been made in view of the conventional art as described above. It is therefore an object of the invention to provide dermatological agents such as whitening agents and hair growth accelerators that contain a compound having high water solubility and a specific structure capable of releasing 2,3-dimethoxy-5-methyl-1,4-dihydroxybenzene (reduced form).

### Solution to Problem

The present inventors studied diligently to solve the problems. They have then found that 2,3-dimethoxy-5-methyl-1,4-dihydroxybenzenecarboxylic acid ester derivatives or salts thereof can solve the problems described above. The present invention has been completed based on the finding.

The present invention is concerned with, for example, the following [1] to [4].

[1] A dermatological agent comprising a 2,3-dimethoxy-5-methyl-1,4-dihydroxybenzenecarboxylic acid ester derivative represented by the following general Formula (I) or a salt thereof:

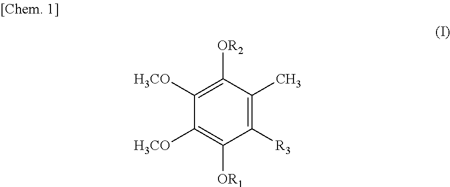

wherein in the Formula (I), OR₁ and OR₂ are each independently a hydroxyl group or a group formed by esterification reaction between a hydroxyl group and a carboxyl or carboxylate group of amino acid, N-acylamino acid, N-alkylamino acid, N,N-dialkylamino acid, pyridinecarboxylic acid or a salt thereof;

OR₁ and OR₂ are not the hydroxyl groups at the same time; and

R₃ is a group represented by Formula (A) below:

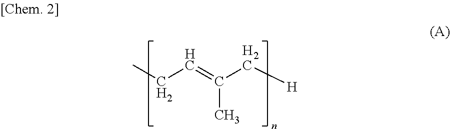

wherein in the Formula (A), n is an integer of 1 to 10.

[2] The dermatological agent as described in [1], which is used as a whitening agent.

[3] The dermatological agent as described in [1], which is used as a hair growth accelerator.

[4] The dermatological agent as described in [1], which is used as an antioxidant.

### ADVANTAGEOUS EFFECTS OF INVENTION

The 2,3-dimethoxy-5-methyl-1,4-dihydroxybenzenecarboxylic acid ester derivatives represented by the general Formula (I) or salts thereof have high water solubility and show effects of inhibiting the melanin production and accelerating hair growth. Therefore, the compounds are useful as whitening ingredients or hair growth accelerating ingredients in a wide range of formulations including cosmetics, quasi drugs and drugs. The dermatological agents according to the present invention contain these compounds and have excellent whitening effects and hair growth accelerating effects.

### BRIEF DESCRIPTION OF DRAWINGS

FIG. 1 shows the results of evaluation of inhibitory effects for melanocyte dendrite formation in Example 1. In FIG. 1, UqH-4-DMG and UqH-1,4-DMG indicate compounds 5 and 4 in Table 1, respectively.

FIG. 2 shows the results of evaluation of inhibitory effects for melanin synthesis in Example 2. In FIG. 2, UqH-4-DMG and UqH-1,4-DMG indicate compounds 5 and 4 in Table 1, respectively.

FIG. 3 shows the results of evaluation of accelerating effects for mouse body hair in Example 3. In FIG. 3, UqH-4-DMG and UqH-1,4-DMG indicate compounds 5 and 4 in Table 1, respectively.

### BEST MODE FOR CARRYING OUT THE INVENTION

Preferred embodiments of the present invention will be described below.

A dermatological agent according to the present invention is characterized by containing a 2,3-dimethoxy-5-methyl-1,4-dihydroxybenzenecarboxylic acid ester derivative represented by the following general Formula (I) or a salt thereof.

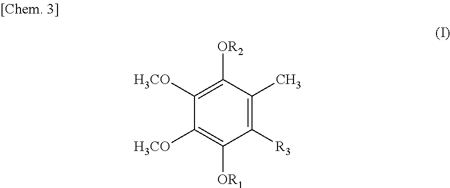

In the Formula (I), OR₁ and OR₂ are each independently a hydroxyl group or a group formed by esterification reaction between a hydroxyl group and a carboxyl or carboxylate group of amino acid, N-acylamino acid, N-alkylamino acid, N,N-dialkylamino acid, pyridinecarboxylic acid or a salt thereof; and OR₁ and OR₂ are not the hydroxyl groups at the same time.

In the present specification, the amino acids indicate organic compounds which have at least one unsubstituted amino group and at least one unsubstituted carboxyl group in the molecule. In the present specification, the amino groups include imino groups (divalent groups represented by ═NR or —NR— wherein R is a hydrogen atom or a substituent group).

In the amino acids, the amino group and the carboxyl group are preferably linked together via a C1-7 linear, branched or cyclic alkylene group. The branched alkylene groups may refer to alkylene groups derived from alkyl groups such as isopropyl, isobutyl and 1-ethylpropyl. The cyclic alkylene groups may refer to alkylene groups having a cyclopentane ring, a cyclohexane ring or a methylcyclohexane ring in the structure. Particularly preferred alkylene groups are methylene and ethylene groups.

From the viewpoints of bioaffinity and water solubility of the 2,3-dimethoxy-5-methyl-1,4-dihydroxybenzenecarboxylic acid ester derivatives or salts thereof contained in the dermatological agents of the present invention, glycine and alanine are preferable, and glycine is more preferable as the amino acid.

The N-acylamino acids are N-acyl compounds of the amino acids. The acyl groups in the N-acylamino acids include C2-6 linear or branched acyl groups. Examples of the acyl groups include acetyl group, n-propanoyl group, n-butanoyl group and tert-butoxycarbonyl group. From the viewpoint of water solubility of the 2,3-dimethoxy-5-methyl-1,4-dihydroxybenzenecarboxylic acid ester derivatives or salts thereof contained in the dermatological agents of the present invention, the acetyl group is preferable as the acyl group. The N-acylamino acids do not have to have an unsubstituted amino group.

The N-alkylamino acids and the N,N-dialkylamino acids are N-alkyl compounds and N,N-dialkyl compounds of the amino acids, respectively. The alkyl groups in these compounds include C1-6 linear or branched alkyl groups. Examples of the alkyl groups include methyl group, ethyl group, n-propyl group, n-butyl group, n-pentyl group, n-hexyl group, isopropyl group, isobutyl group, 1-methylpropyl group, tert-butyl group, 1-ethylpropyl group and isoamyl group. Among them, from the viewpoint of water solubility of the 2,3-dimethoxy-5-methyl-1,4-dihydroxybenzenecarboxylic acid ester derivatives or salts thereof contained in the dermatological agents of the present invention, alkyl groups of 3 or less carbon atoms are preferable, and the methyl and ethyl groups are particularly preferable. The N-alkylamino acids and the N,N-dialkylamino acids may not have an unsubstituted amino group.

The salts of the amino acids, N-acylamino acids, N-alkylamino acids, N,N-dialkylamino acids or pyridinecarboxylic acids are preferably hydrogen halide salts or alkylsulfonic acid salts. When OR₁ and OR₂ are groups formed by esterification reaction between a hydroxyl group and a carboxylate group of a hydrogen halide salt of, for example, amino acid, the 2,3-dimethoxy-5-methyl-1,4-dihydroxybenzenecarboxylic acid ester derivative of the general Formula (I) is in the form of salt as well. The salts usually have a higher melting point than the original quinone compounds, and thereby permit easy handling in formulation. Preferred hydrogen halide salts are hydrochlorides and hydrobromides. Examples of the alkylsulfonic acid salts include methanesulfonic acid salts.

In the general Formula (I), R₃ is a group represented by Formula (A) below.

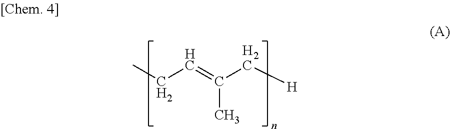

In the Formula (A), n is an integer of 1 to 10, and preferably an integer of 6 to 10.

The compounds represented by the general Formula (I) may be produced by various processes. A representative process will be described below. A 2,3-dimethoxy-5-methyl-1,4-benzoquinone compound represented by the general Formula (II) below is reduced with a reducing agent to give a 2,3-dimethoxy-5-methyl-1,4-dihydroxybenzene compound represented by the general Formula (III) below.

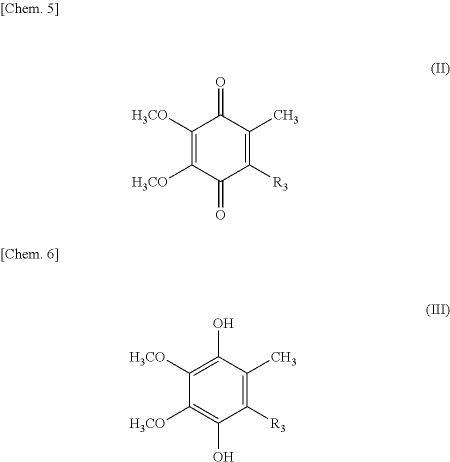

In the general Formulae (II) and (III), R³ is the same as R³ in the general Formula (I). The reducing agents used herein include sodium borohydride, sodium hydrosulfite, tri-n-butylphosphine, zinc chloride and stannous chloride.

The 2,3-dimethoxy-5-methyl-1,4-dihydroxybenzene compound is then esterified with an amino acid, N-acylamino acid, N-alkylamino acid, N,N-dialkylamino acid, pyridinecarboxylic acid, a salt of these acids or a reactive acid derivative thereof according to a common method, thereby affording a compound represented by the general Formula (I).

In the production of the 2,3-dimethoxy-5-methyl-1,4-dihydroxybenzenecarboxylic acid ester derivatives for the dermatological agents of the present invention, good results maybe obtained by protecting the amino group or secondary amino group of the amino acid or respective functional groups present in side chains of the amino acid with appropriate protective groups such as tert-butoxycarbonyl group (hereinafter, abbreviated to t-BOC group), benzyloxycarbonyl group (hereinafter, abbreviated to Z group) and 9-fluorenylmethoxycarbonyl group (hereinafter, abbreviated to FMOC group), and carrying out the esterification reaction in the presence of an active esterification reagent such as dicyclohexylcarbodiimide (hereinafter, abbreviated to DCC), 1-methyl-3-(3-dimethylaminopropyl)-carbodiimide hydrochloride (hereinafter, abbreviated to EDC) or N,N-disuccinimide oxalate (hereinafter, abbreviated to DSO).

In the case of the N,N-dialkylamino acid or pyridinecarboxylic acid, favorable results may be obtained by using a hydrogen halide salt thereof and carrying out the esterification reaction in the presence of an active esterification reagent such as DCC, EDC or DSO. In this case, pyridine is preferably used as a reaction solvent.

When the reactive acid derivative is used, preferred results may be obtained by carrying out the esterification reaction (the reaction with the 2,3-dimethoxy-5-methyl-1,4-dihydroxybenzene compound), with the carboxyl or carboxylate group of the amino acid or the like activated with an acid halogenite, in particular an acid chloride. In this case, an anhydrous benzene/anhydrous pyridine mixture is preferably used as a solvent.

The hydrogen halide salts and the alkylsulfonic acid salts of the 2,3-dimethoxy-5-methyl-1,4-dihydroxybenzenecarboxylic acid ester derivatives contained in the dermatological agents of the invention, may be produced by reacting the free 2,3-dimethoxy-5-methyl-1,4-dihydroxybenzenecarboxylic acid ester derivative with a hydrogen halide or an alkylsulfonic acid according to a common process.

In the case where the protective groups have been introduced into the amino acid or the like in the production of the 2,3-dimethoxy-5-methyl-1,4-dihydroxybenzenecarboxylic acid ester derivative, deprotecting the compound with a hydrohalic acid according to a common process gives rise to the deprotection and at the same time, production of the corresponding hydrogen halide.

The compounds or salts thereof represented by the general Formula (I) possess whitening effects and show marked inhibitory effects in melanocyte growth and melanin formation inhibitory tests using a three dimensional human skin culture model.

In addition, the compounds or salts thereof represented by the general Formula (I) possess hair growth accelerating effects and show marked effects in hair growth stimulation in the testing of hair growth accelerating effects using C3H mice.

Further, the compounds or salts thereof represented by the general Formula (I) have high water solubility and are easily formulated in aqueous dermatological agents. As a result, there is no need for separately adding additives such as surfactants to solubilize the compounds and salts thereof. The compounds are thus advantageous in terms of formulation and safety.

In the present specification, the term “whitening effects” should be interpreted in the broadest sense including not only inhibitory effects against melanin formation but also other effects such as inhibition of pigmentation, and prevention and improvements of dull skin and tanned skin due to sunburn or the like.

A dermatological agent according to the present invention contains the 2,3-dimethoxy-5-methyl-1,4-dihydroxybenzenecarboxylic acid ester derivative or salt thereof represented by the above general Formula (I). The content thereof is preferably 0.00001 to 6% by mass, and more preferably 0.01 to 1% by mass of the whole dermatological agent (100% by mass). The content in this range ensures that the compound is stably formulated in the dermatological agent and the obtainable dermatological agent shows excellent medicinal effects.

The formulation forms of the dermatological agents of the present invention are not particularly limited. Examples include emulsions, creams, lotions, packs, cleansers, dispersions, ointments, detergents, aerosols, adhesive skin patches, cataplasms and liniments. The dermatological agents may be cosmetics or external medicines in any of these forms.

Where necessary, the dermatological agents of the present invention may contain ingredients commonly used in formulations such as cosmetics, quasi drugs and external medicines, while still achieving the advantageous effects of the present invention. Examples of such ingredients include water, alcohols, oil agents, surfactants, metallic soaps, gelatinizers, powders, alcohols, water soluble polymers, film-forming agents, resins, UV protective agents, clathrate compounds, antimicrobial agents, perfumes, deodorants, salts, pH regulators, algefacients, animal/microbial deriving extracts, plant extracts, blood circulation promoters, astringents, antiseborrheic agents, whitening agents, anti-inflammatory agents, active oxygen scavengers, cell activators, moisturizers, chelating agents, keratolytic agents, rinsing agents, enzymes, hormones and vitamins.

### EXAMPLES

The present invention will be described in detail by presenting examples hereinbelow without limiting the scope of the present invention.

### Synthetic Examples

2,3-Dimethoxy-5-methyl-1,4-dihydroxybenzene derivatives and salts thereof shown in Tables 1 and 3 were produced by the following production processes A to D.

Production Process A

0.1 Mol of amino acid was dissolved in 100 ml of a distilled water/dioxane mixture (1:1, v/v), and 30 ml of triethylamine was added thereto. Di-tert-butyl dicarbonate was gradually added. The mixture was stirred at room temperature for 30 minutes.

The dioxane was distilled away under reduced pressure. 50 ml of an aqueous sodium hydrogen carbonate solution (0.5 M) was added, and the mixture was washed with 100 ml of ethyl acetate. The ethyl acetate layer was washed with 50 ml of an aqueous sodium hydrogen carbonate solution. The aqueous layers were combined and were rendered acidic (pH 3) by adding an aqueous citric acid solution (0.5 M) under ice cooling. The solution was then saturated with sodium chloride and was extracted with ethyl acetate (100 ml×3).

The extract liquid was dehydrated over anhydrous sodium sulfate, and the solvent was distilled away under reduced pressure. The oily residue was crystallized by the addition of isopropyl ether or by cooling, thereby producing N-t-BOC-amino acid.

1.16 mmol of 2,3-dimethoxy-5-methyl-6-decaisoprenyl-1,4-benzoquinone (ubiquinone-10) was dissolved in 100 ml of isopropyl ether. A suspension of 2.8 mmol of sodium borohydride in 15 ml of methanol was added. The yellow solution was stirred at room temperature until the solution became colorless.

To the reaction liquid, 100 ml of distilled water saturated with argon gas was added to wash the isopropyl ether layer. After liquid separation was carried out, the isopropyl ether layer was dehydrated over anhydrous sodium sulfate. The solvent was distilled away under reduced pressure to give 2,3-dimethoxy-5-methyl-6-decaisoprenyl-1,4-dihydroxybenzene (ubiquinol-10).

2.8 mmol of N-t-BOC-amino acid, 2.8 mmol of DCC and 30 ml of anhydrous pyridine were added to the 2,3-dimethoxy-5-methyl-6-decaisoprenyl-1,4-dihydroxybenzene. The atmosphere was replaced with argon gas, and the mixture was stirred at room temperature for 24 hours.

The solvent was distilled away under reduced pressure. Ethyl acetate was added to the residue to extract soluble fractions (100 ml×twice). The extract liquid was concentrated under reduced pressure, and the residue was separated and purified by silica gel flash chromatography (eluting solvent; n-hexane:ethyl acetate=85:15) to give 2,3-dimethoxy-5-methyl-6-decaisoprenyl-benzene 1,4-bis-N-t-BOC-amino acid ester.

The 2,3-dimethoxy-5-methyl-6-decaisoprenylbenzene 1,4-bis-N-t-BOC-amino acid ester was dissolved in a small amount of acetone. Hydrochloric acid-dioxane (3.5 N) in an amount corresponding in terms of hydrochloric acid to approximately 20 times the amount of the ester bonds was added to perform deprotection. After the completion of the reaction, the solvent was distilled away under reduced pressure and the residue was recrystallized with acetone to give 2,3-dimethoxy-5-methyl-6-decaisoprenylbenzene-1,4-bis-amino acid ester hydrochloride.

Production Process B

1.16 mmol of 2,3-dimethoxy-5-methyl-6-decaisoprenyl-1,4-benzoquinone (ubiquinone-10) was dissolved in 100 ml of isopropyl ether. A suspension of 2.8 mmol of sodium borohydride in 15 ml of methanol was added. The yellow solution was stirred at room temperature until the solution became colorless.

To the reaction liquid, 100 ml of distilled water saturated with argon gas was added to wash the isopropyl ether layer. After liquid separation was carried out, the isopropyl ether layer was dehydrated over anhydrous sodium sulfate. The solvent was distilled away under reduced pressure to give 2,3-dimethoxy-5-methyl-6-decaisoprenyl-1,4-dihydroxybenzene (ubiquinol-10).

1.4 mmol of N-t-BOC-amino acid synthesized in Production Process A, 1.4 mmol of DCC and 30 ml of anhydrous pyridine were added to the 2,3-dimethoxy-5-methyl-6-decaisoprenyl-1,4-dihydroxybenzene. The atmosphere was replaced with argon gas, and the mixture was stirred at room temperature for 24 hours. The solvent was distilled away under reduced pressure. Ethyl acetate was added to the residue to extract soluble fractions (100 ml×twice).

The extract liquid was concentrated under reduced pressure, and the residue was separated and purified by silica gel flash chromatography (eluting solvent; n-hexane:ethyl acetate=85:15) to give 2,3-dimethoxy-5-methyl-6-decaisoprenyl-4-hydroxybenzene 1-N-t-BOC-amino acid ester and 2,3-dimethoxy-5-methyl-6-decaisoprenyl-1-hydroxybenzene 4-N-t-BOC-amino acid ester.

The 2,3-dimethoxy-5-methyl-6-decaisoprenyl-4-hydroxybenzene 1-N-t-BOC-amino acid ester or 2,3-dimethoxy-5-methyl-6-decaisoprenyl-1-hydroxybenzene 4-N-t-BOC-amino acid ester was dissolved in a small amount of acetone. Hydrochloric acid-dioxane (3.5 N) in an amount corresponding in terms of hydrochloric acid to approximately 20 times the amount of the ester bonds was added to perform deprotection.

After the completion of the reaction, the solvent was distilled away under reduced pressure and the residue was recrystallized with acetone to give 2,3-dimethoxy-5-methyl-6-decaisoprenyl-4-hydroxybenzene 1-amino acid ester hydrochloride and 2,3-dimethoxy-5-methyl-6-decaisoprenyl-1-hydroxybenzene 4-amino acid ester hydrochloride.

Production Process C

1.16 mmol of 2,3-dimethoxy-5-methyl-6-decaisoprenyl-1,4-benzoquinone (ubiquinone-10) was dissolved in 100 ml of isopropyl ether. A suspension of 2.8 mmol of sodium borohydride in 15 ml of methanol was added. The yellow solution was stirred at room temperature until the solution became colorless.

To the reaction liquid, 100 ml of distilled water saturated with argon gas was added to wash the isopropyl ether layer. After liquid separation was carried out, the isopropyl ether layer was dehydrated over anhydrous sodium sulfate. The solvent was distilled away under reduced pressure to give 2,3-dimethoxy-5-methyl-6-decaisoprenyl-1,4-dihydroxybenzene (ubiquinol-10).

2.8 mmol of N,N-dialkylamino acid hydrochloride, 2.8 mmol of DCC and 30 ml of anhydrous pyridine were added to the 2,3-dimethoxy-5-methyl-6-decaisoprenyl-1,4-dihydroxybenzene. The atmosphere was replaced with argon gas, and the mixture was stirred at room temperature for 24 hours.

The solvent was distilled away under reduced pressure. The residue was suspended in distilled water, and pH of the suspension was adjusted to 7 to 8 by the addition of sodium hydrogen carbonate. Ethyl acetate was added to extract soluble fractions (100 ml×3 times) . The extract liquid was dehydrated with anhydrous sodium sulfate, and the solvent was distilled away under reduced pressure. The residue was separated and purified by silica gel flash chromatography (eluting solvent; n-hexane:ethyl acetate=85:15) to give 2,3-dimethoxy-5-methyl-6-decaisoprenylbenzene 1,4-bis-N,N-dialkylamino acid ester.

The 2,3-dimethoxy-5-methyl-6-decaisoprenylbenzene 1,4-bis-N,N-dialkylamino acid ester was dissolved in a small amount of n-hexane. Hydrochloric acid-dioxane in a two-fold molar amount was added. The solvent was distilled away under reduced pressure and the residue was recrystallized with acetone to give 2,3-dimethoxy-5-methyl-6-decaisoprenylbenzene 1,4-bis-N,N-dialkylamino acid ester hydrochloride.

Production Process D

1.16 mmol of 2,3-dimethoxy-5-methyl-6-decaisoprenyl-1,4-benzoquinone (ubiquinone-10) was dissolved in 100 ml of isopropyl ether. A suspension of 2.8 mmol of sodium borohydride in 15 ml of methanol was added. The yellow solution was stirred at room temperature until the solution became colorless.

To the reaction liquid, 100 ml of distilled water saturated with argon gas was added to wash the isopropyl ether layer. After liquid separation was carried out, the isopropyl ether layer was dehydrated over anhydrous sodium sulfate. The solvent was distilled away under reduced pressure to give 2,3-dimethoxy-5-methyl-6-decaisoprenyl-1,4-dihydroxybenzene (ubiquinol-10).

2.8 mmol of N,N-dialkylamino acid hydrochloride, 2.8 mmol of DCC and 30 ml of anhydrous pyridine were added to the 2,3-dimethoxy-5-methyl-6-decaisoprenyl-1,4-dihydroxybenzene. The atmosphere was replaced with argon gas, and the mixture was stirred at room temperature for 24 hours. The solvent was distilled away under reduced pressure. The residue was suspended in distilled water, and pH of the suspension was adjusted to 7 to 8 by the addition of sodium hydrogen carbonate. Ethyl acetate was added to extract soluble fractions (100 ml×3 times) . The extract liquid was dehydrated over anhydrous sodium sulfate, and the solvent was distilled away under reduced pressure to give 2,3-dimethoxy-5-methyl-6-decaisoprenyl-4-hydroxybenzene 1-N,N-dialkylamino acid ester and 2,3-dimethoxy-5-methyl-6-decaisoprenyl-1-hydroxybenzene 4-N,N-dialkylamino acid ester.

The 2,3-dimethoxy-5-methyl-6-decaisoprenyl-4-hydroxybenzene 1-N,N-dialkylamino acid ester or 2,3-dimethoxy-5-methyl-6-decaisoprenyl-1-hydroxybenzene 4-N,N-dialkylamino acid ester was dissolved in a small amount of n-hexane. Hydrochloric acid-dioxane in a two-fold molar amount was added. The solvent was distilled away under reduced pressure and the residue was recrystallized with acetone to give 2,3-dimethoxy-5-methyl-6-decaisoprenyl-4-hydroxybenzene 1-N,N-dialkylamino acid ester hydrochloride and 2,3-dimethoxy-5-methyl-6-decaisoprenyl-1-hydroxybenzene 4-N,N-dialkylamino acid ester hydrochloride.

2,3-Dimethoxy-5-methyl-6-decaisoprenylbenzene 1,4-bis-N-alkylamino acid ester hydrochloride, 2,3-dimethoxy-5-methyl-6-decaisoprenyl-4-hydroxybenzene 1-N-alkylamino acid ester hydrochloride and 2,3-dimethoxy-5-methyl-6-decaisoprenyl-1-hydroxybenzene 4-N-alkylamino acid ester hydrochloride were producible in accordance with Production Processes A and B, in detail in accordance with Production Process A except that the amino acid was replaced by N-alkylamino acid (the reaction of the N-alkylamino acid with di-tert-butyl dicarbonate would give a protected product, which is here referred to as N-alkyl-N-t-BOC amino acid), or in accordance with Production

Process B except that the N-t-BOC amino acid was replaced by N-alkyl-N-t-BOC amino acid.

Tables 1 and 3 below set forth the specific chemical formulae, properties and production processes of the compounds according to the present invention. Table 2 shows mass spectroscopy data (m/z, FAB-MS) and nuclear magnetic resonance spectra (¹H-NMR, δ (ppm, internal standard TMS)) of compounds with the compound numbers 1 to 6.

TABLE 1

| | Compound Name | | | | | Melting | Production |
|---|---|---|---|---|---|---|---|
| Compound No. | R₁ | R₂ | R₃ | Salt | Shape | Point | Process |
| 1 | 2,3-dimethoxy-5-methyl-6-decaisoprenylbenzene | | | | | | |
| | 1,4-bis-sarcosinate hydrochloride | | | | | | |
| | CH₃NHCH₂CO— | CH₃NHCH₂CO— | 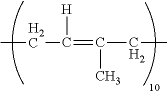 | HCl | white crystal | 210-213 | A |
| 2 | 2,3-dimethoxy-5-methyl-6-decaisoprenyl-1- | | | | | | |
| | hydroxybenzene 4-sarcosinate hydrochloride | | | | | | |
| | H— | CH₃NHCH₂CO— | 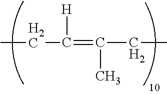 | HCl | white crystal | 110-112 | B |
| 3 | 2,3-dimethoxy-5-methyl-6-decaisoprenyl-4- | | | | | | |
| | hydroxybenzene 1-sarcosinate hydrochloride | | | | | | |
| | CH₃NHCH₂CO— | H— | 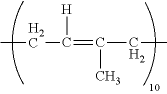 | HCl | white crystal | 95-98 | B |
| 4 | 2,3-dimethoxy-5-methyl-6-decaisoprenylbenzene | | | | | | |
| | 1,4-bis-N,N-dimethyl glycinate hydrochloride | | | | | | |
| | (CH₃)₂NCH₂CO— | (CH₃)₂NCH₂CO— | 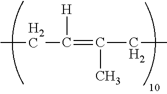 | HCl | white crystal | 179-181 | C, D |
| 5 | 2,3-dimethoxy-5-methyl-6-decaisoprenyl-1- | | | | | | |
| | hydroxybenzene 4-N,N-dimethyl glycinate | | | | | | |
| | hydrochloride | | | | | | |
| | H— | (CH₃)₂NCH₂CO— | 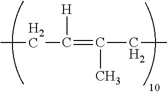 | HCl | white crystal | 75-78 | C, D |
| 6 | 2,3-dimethoxy-5-methyl-6-decaisoprenyl-4- | | | | | | |
| | hydroxybenzene 1-N,N-dimethyl glycinate | | | | | | |
| | hydrochloride | | | | | | |
| | (CH₃)₂NCH₂CO— | H— | 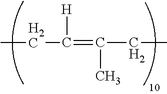 | HCl | white crystal | 82-85 | C, D |

TABLE 2

| Compound | | |
|---|---|---|
| No. | Mass Spectroscopy | ¹H—NMR Spectrum |
| | | (In CDCl₃) |
| 1 | 1007 (M − 2HCl + H⁺) | 5.10 (9H, m), 4.88 (1H, t), 4.15 (2H, s), 4.12 (2H, s), |
| | | 3.70 (3H, s), 3.68 (3H, s), 3.14 (2H, d), 2.86 (6H, s), |
| | | 3.12~1.88 (39H, m), 1.76~1.57 (33H, m) |
| 2 | 936 (M − HCl + H⁺) | 5.70 (1H, s), 5.10 (10H, m), 4.15 (2H, s), 3.87 (3H, s), |
| | | 3.81 (3H, s), 3.31 (2H, d), 2.85 (3H, s), 2.13~1.94 |
| | | (39H, m), 1.76~1.57 (33H, m) |
| 3 | 936 (M − HCl + H⁺) | 5.73 (1H, s), 5.11 (10H, m), 4.08 (2H, s), 3.87 (3H, s), |
| | | 3.81 (3H, s), 3.15 (2H, d), 2.84 (3H, s), 2.13~1.94 |
| | | (39H, m), 1.76~1.57 (33H, m) |
| 4 | 1035 (M − 2HCl + H⁺) | 5.09 (9H, m), 4.89 (1H, t), 4.29 (2H, s), 4.20 (2H, s), |
| | | 3.84 (6H, s), 3.21 (2H, d), 3.11 (6H, s), 3.07 (6H, s), |
| | | 2.09~1.94 (39H, m), 1.73~1.58 (33H, m) |
| 5 | 950 (M − HCl + H⁺) | 5.74 (1H, s), 5.09 (10H, m), 4.18 (2H, s), 3.90 (3H, s), |
| | | 3.82 (3H, s), 3.34 (2H, d), 3.09 (6H, s), 2.09~1.94 |
| | | (39H, m), 1.76~1.58 (33H, m) |
| 6 | 950 (M − HCl + H⁺) | 5.77 (1H, s), 5.11 (9H, m), 4.91 (1H, s), 4.11 (2H, s), |
| | | 3.90 (3H, s), 3.83 (3H, s), 3.17 (2H, d), 3.06 (6H, s), |
| | | 2.14 (3H, s), 2.09~1.93 (36H, m), 1.72~1.57 (33H, m) |

TABLE 3

| | Compound Name | | | | Mass | Production |
|---|---|---|---|---|---|---|
| Compound No. | R₁ | R₂ | R₃ | Shape | Spectroscopy | Process |
| 7 | 2,3-dimethoxy-5-methyl-6-decaisoprenylbenzene 1,4-bis-N-t-BOC- | | | | | |
| | sarcosinate | | | | | |
| | N-t-BOC-N(CH₃)CH₂CO— | N-t-BOC-N(CH₃)CH₂CO— | 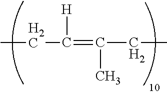 | oily | 1207 | A |
| 8 | 2,3-dimethoxy-5-methyl-6-decaisoprenyl-1-hydroxybenzene | | | | | |
| | 4-N-t-BOC-sarcosinate | | | | | |
| | H— | N-t-BOC-N(CH₃)CH₂CO— | 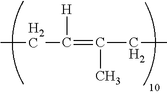 | oily | 1036 | B |
| 9 | 2,3-dimethoxy-5-methyl-6-decaisoprenyl-4-hydroxybenzene | | | | | |
| | 1-N-t-BOC-sarcosinate | | | | | |
| | N-t-BOC-N(CH₃)CH₂CO— | H— | 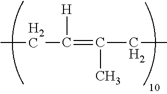 | oily | 1036 | B |
| 10 | 2,3-dimethoxy-5-methyl-6-decaisoprenylbenzene 1,4-bis-sarcosinate | | | | | |
| | CH₃NHCH₂CO— | CH₃NHCH₂CO— | 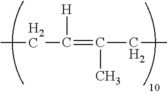 | oily | 1007 | A |
| 11 | 2,3-dimethoxy-5-methyl-6-decaisoprenyl-1-hydroxybenzene 4-sarcosinate | | | | | |
| | H— | CH₃NHCH₂CO— | 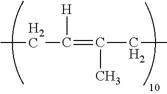 | oily | 936 | B |
| 12 | 2,3-dimethoxy-5-methyl-6-decaisoprenyl-4-hydroxybenzene 1-sarcosinate | | | | | |
| | CH₃NHCH₂CO— | H— | 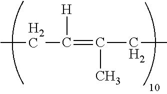 | oily | 936 | B |
| 13 | 2,3-dimethoxy-5-methyl-6-decaisoprenylbenzene 1,4-bis-N,N-dimethyl glycinate | | | | | |
| | (CH₃)₂NHCH₂CO— | (CH₃)₂NHCH₂CO— | 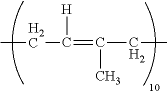 | oily | 1035 | C |
| 14 | 2,3-dimethoxy-5-methyl-6-decaisopreny-1-hydroxybenzene | | | | | |
| | 4-N,N-dimethyl glycinate | | | | | |
| | H— | (CH₃)₂NHCH₂CO— | 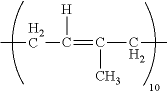 | oily | 950 | D |
| 15 | 2,3-dimethoxy-5-methyl-6-decaisoprenyl-4-hydroxybenzene | | | | | |
| | 1-N,N-dimethyl glycinate | | | | | |
| | (CH₃)₂NHCH₂CO— | H— | 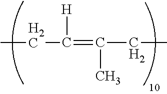 | oily | 950 | D |

### Example 1

### Inhibitory Effects For Melanocyte Dendrite Formation

Melanin formation inhibitory effects of the compounds 4 and 5 shown in Table 1 were evaluated using three dimensional human skin model MEL-300 (normal human skin keratinocytes containing normal human epidermal melanocytes). The MEL-300 skin model cups were transferred to a 6-well plate, each well containing 0.9 ml of EPI-100-LLMM maintenance medium, and were pre-incubated at 37° C. and 5% CO₂ for 1 hour.

The maintenance medium was suction removed, and 5 ml of the maintenance medium was newly added. Thereafter, 0.1 ml of a 30 mmol/l solution of the compound 4 or 5 was added to the MEL-300 skin model cups, and the resultant were incubated. The controls were given 0.1 ml of a 0.2% ethanol solution.

The maintenance medium and the test substance were replaced every two days, and the incubation was performed for 7 days. After the 7 days of incubation, the cells inside the three dimensional human skin model cup were photographed, and the proportion (%) of the area of melanocytes was calculated by image analysis. Significant difference was tested by a post-hoc test (Scheffe method). The significance level was 5%. The results are set forth in FIG. 1. As clearly shown in FIG. 1, the compounds 4 and 5 significantly inhibited the formation of melanocyte dendrites. In this experiment, n=3.

### Example 2

### Melanin Synthesis Inhibitory Test

Melanin formation inhibitory effects of the compounds 4 or 5 shown in Table 1 were evaluated using three dimensional human skin model MEL-300 (normal human skin keratinocytes containing normal human epidermal melanocytes). The MEL-300 skin model cups were transferred to a 6-well plate, each well containing 0.9 ml of EPI-100-LLMM maintenance medium, and were pre-incubated at 37° C. and 5% CO₂ for 1 hour.

The maintenance medium was suction removed, and 5 ml of the maintenance medium was newly added. Thereafter, 0.1 ml of a 30 mmol/l solution of the compound 4 or 5 was added to the MEL-300 skin model cups, and the resultant were incubated. The controls were given 0.1 ml of a 0.2% ethanol solution. The positive controls were given 0.1 ml of a 30 mmol/l kojic acid solution.

The maintenance medium and the test substance were replaced every two days, and the incubation was performed for 14 days. After the 14 days of incubation, the incubated skin pieces were taken out from the MEL-300 skin model cups and were each impregnated with 0.2 ml of a 1% aqueous SDS solution containing 0.05 mM EDTA and 1% tris-hydrochloric acid solution. Further, 0.02 ml of a 5 mg/mL proteinase K solution was added, and the reaction was performed at 37° C. overnight.

After the reaction, the reaction liquid was sufficiently stirred and the incubated skin piece was completely crushed. To the reaction liquid, 0.025 ml of a 500 mM sodium carbonate solution and 0.005 ml of a 30% hydrogen peroxide solution were added followed by heating at 80° C. for 30minutes. After cooling, 0.1 ml of a chloroform/methanol solution (2:1 (vol/vol)) was added, and the mixture was centrifuged. The supernatant was analyzed to determine the absorbance at 405 nm. A linear calibration curve was prepared based on the absorbances of melanin solutions of known concentrations that had been treated similarly. The melanin contents in the incubated skin pieces were thus determined.

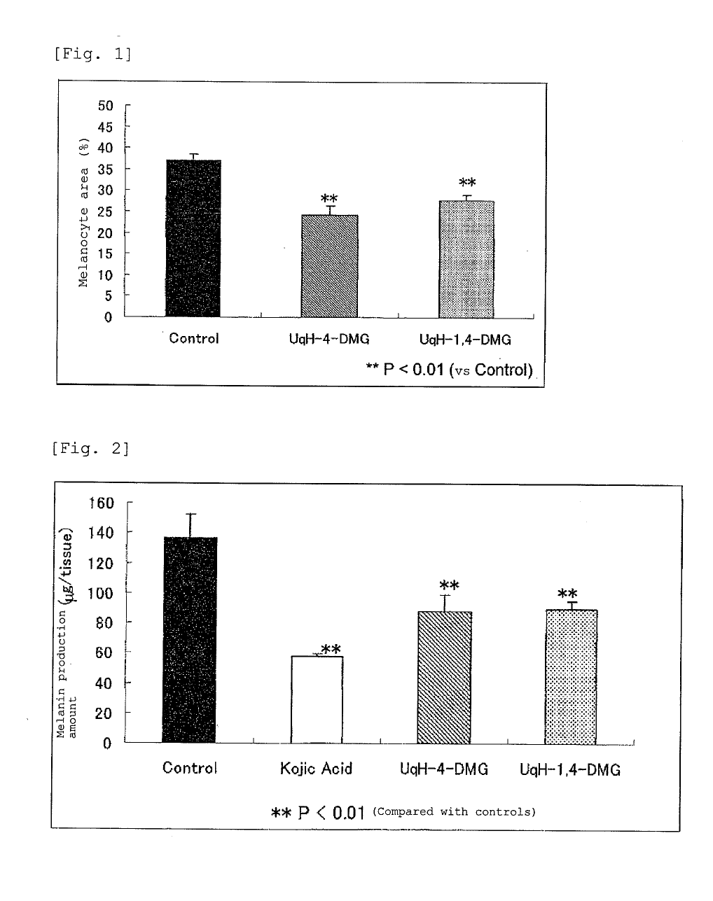

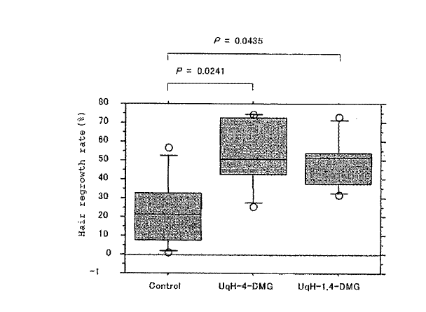

Significant difference was tested by a post-hoc test (Scheffe method). The significance level was 1%. The melanin production was significantly decreased by the addition of the compound 4 or 5 to the three dimensional human skin model (FIG. 2). This result showed that the compounds 4 and 5 had melanin formation inhibitory effects and functioned as whitening agents. In this experiment, n=3.

### Example 3

### Accelerating Effects For Mouse Body Hair Growth

Male C3H/HeN that were seven weeks old in a telogen hair cycle were used.

The dorsal region of the mice was shaved with an electrical hair clipper (0.05 mm blades) and an electrical shaver. From the next day, 0.1 ml of a 50 μmol/g ointment of the compound 4 or 5 was coated on the shaved regions once a day using a bacteria spreader. The controls were given only coating stimulation with a bacteria spreader.

The dorsal region of the mice on Day 18 after the initiation of coating was photographed. The proportion of the area of the region where hair had regrown relative to the total area of the shaved region was calculated by image analysis. The value obtained thereby indicated the hair regrowth rate (%). Significant difference was tested by a post-hoc test (Scheffe method). The significance level was 5%.

The hair regrowth rate (%) was significantly increased by coating the ointment containing the compound 4 or 5 (FIG. 3). This result revealed that the compounds 4 and 5 had effects of accelerating the body hair growth. In this experiment, n=6 for the test substances and n=7 for the controls.

1. A dermatological agent comprising a 2,3-dimethoxy-5-methyl-1,4-dihydroxybenzenecarboxylic acid ester derivative represented by the following general Formula (I) or a salt thereof: 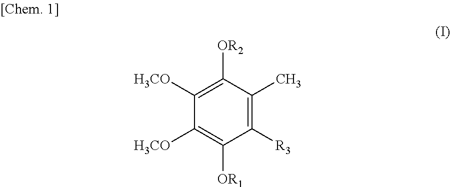
wherein in the Formula (I), OR₁ and OR₂ are each independently a hydroxyl group or a group formed by esterification reaction between a hydroxyl group and a carboxyl or carboxylate group of amino acid, N-acylamino acid, N-alkylamino acid, N,N-dialkylamino acid, pyridinecarboxylic acid or a salt thereof;
OR₁ and OR₂ are not the hydroxyl groups at the same time; and
R₃ is a group represented by Formula (A) below: 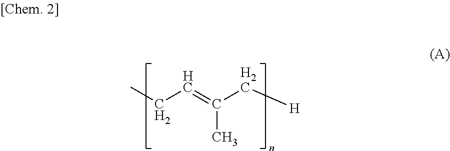
wherein n is an integer of 1 to 10.

2. The dermatological agent according to claim 1, which is used as a whitening agent.

3. The dermatological agent according to claim 1, which is used as a hair growth accelerator.

4. The dermatological agent according to claim 1, which is used as an antioxidant.
